(19) United States
(12) Patent Application Publication
(10) Pub. No.: US 2025/0164443 A1
(43) Pub. Date: May 22, 2025

(54) MEASURING IONS WITH CHARGE REDUCTION

(71) Applicant: Micromass UK Limited, Wilmslow (GB)

(72) Inventors: Jakub Ujma, Wilmslow (GB); Kevin Giles, Wilmslow (GB)

(73) Assignee: Micromass UK Limited, Wilmslow (GB)

(21) Appl. No.: 18/841,235

(22) PCT Filed: Feb. 24, 2023
(86) PCT No.: PCT/GB2023/050421

§ 371 Date: Aug. 23, 2024

(30) Foreign Application Priority Data
Feb. 25, 2022 (GB) 2202665.2
Jun. 1, 2022 (GB) 2208168.1

(51) Int. Cl. G01N 27/623; H01J 49/00

(52) U.S. Cl. CPC G01N 27/623; H01J 49/0036

(57) ABSTRACT

Disclosed are methods of mass spectrometry that use charge reduction techniques to reduce the average charge of the ions to increase the mass to charge ratio spacings between different charge states for the ion species prior to mass analysis. In embodiments there is disclosed a method for determining a collision cross section for an ion species, wherein an ion mobility is determined for an ion species in a first state without charge reduction and the mass to charge ratios for the ion species are then measured in a second, charge-reduced state. The average charge determined for the ion species in the first state is then used together with the average mass determined from the ion species in the second state and the ion mobility determined for the ion species in the first state to determine a collision cross section for the ion species in the first state.

### CROSS-REFERENCES TO RELATED APPLICATIONS

This application claims priority from and the benefit of United Kingdom patent application No. 2202665.2 filed on 25 Feb. 2022 and United Kingdom patent application No. 2208168.1 filed on 1 Jun. 2022. The entire contents of these applications are incorporated herein by reference.

### FIELD

The present disclosure relates generally to mass spectrometry, and in particular to using charge reduction to enable mass determination of high molecular weight (>1 MDa) species. In embodiments, the present disclosure relates to methods of ion mobility spectrometry, and in particular to measuring collision cross-sections of ions that are separated within a drift tube ion mobility spectrometer.

### BACKGROUND

Ion mobility separation is an analytical technique that separates ions on the basis of their size, shape, charge and mass. Ion mobility separation may be used as a stand-alone technique. However, ion mobility separation is often advantageously coupled with mass spectrometry as it extends the information that can be obtained about an ion as it delivers information about the three-dimensional structure of an ion, and increases peak capacity and confidence in compound characterization. Ion mobility-mass spectrometry also offers significant utility in the analysis of isomeric species, where different structures are reflected by differences in collision cross sections.

An ion's collision cross section can therefore be an important distinguishing characteristic of the ion in the gas phase. The collision cross section is related to the ions chemical structure and three-dimensional conformation. Collision cross section can thus be used as an additional molecular identifier to help confirm the identity of an ion or investigate its structure. Moreover, it can also be used to increase confidence in the quantitative measurement of known compounds, thus reducing interferences and improving signal to noise ratios. For this reason, measurements of collision cross section are often desirable during experimental analysis, as the measured collision cross section can be compared to characteristic collision cross section values (include in libraries and databases) to confirm identification of an ion.

Various different types of ion mobility separation devices exist. In classical drift tube ion mobility separation devices, packets of analyte ions travel through a gas-filled “drift tube” under the influence of a uniform electric field and their arrival time is recorded at a detector (which may be a mass spectrometer, but could also be a stand-alone detector). In this type of arrangement, packets of ions move at terminal velocity (‘drift’) through the buffer gas with a drift velocity (v) that is related to the strength of the electric field (E) via mobility (K), according to the following equation:

$$v = KE \qquad ( Equation 1 )$$

The velocity at which the ions drift through the buffer gas within the ion mobility separation device will depend on the instrument operational parameters such as buffer gas temperature and composition but more importantly for separation, also the physicochemical properties of the ion and the gas, including ion charge state, the ion and gas molecule masses, and the rotationally averaged collision cross section of the ion and gas molecules. As mentioned above, the collision cross section is an analytical metric indicative of conformational preferences of the molecule from which the ion is derived. Determining the collision cross section for an ion can therefore be useful in order to classify or identify the ion species.

With knowledge of the physical conditions within the ion mobility separation device and the properties of the ion (i.e. its mass and charge), it is thus possible to convert between the ion's mobility (K), as determined from the recorded drift times, and the ion's collision cross-section (CCS), e.g., and in particular, using the Mason-Schamp equation:

$$K \approx \frac{3}{16} \frac{ze}{N} {( \frac{1}{M} + \frac{1}{M} )}^{1 / 2} {( \frac{1}{{k}_{B} T} )}^{1 / 2} \frac{1}{CCS} \qquad ( Equation 2 )$$

where z is the number of charges on the ion, e is the elementary electric charge, N is the number of gas molecules per unit volume (in the buffer gas), M and m are masses of the ion and the buffer gas molecules, k_{B} is the Boltzmann constant and T corresponds to the temperature of the buffer gas.

Combined ion mobility-mass spectrometry is thus a powerful analytic for characterising ions, as the mass spectrometry can provide the mass and charge values for an ion, and the ion mobility separation can determine the ion mobility (K), which information can together be used to calculate the ion's collision cross-section (CCS) using Mason-Schamp equation (Equation 2).

However, there are still situations where it may be difficult to reliably determine the precise mass and charge values for an ion species, particularly where there is heterogeneity in the mass of different ions corresponding to the same ion species and/or relatively higher numbers of different charge states, which can make it difficult to resolve different mass to charge ratio peaks within the mass spectra using conventional instruments, and where the above approach may therefore not work well. This may especially be the case for larger biomolecules including high mass protein complexes such as virus capsids where there may be a high level of adduction and heterogeneity in the virus protein ratio yielding broad distributions of overlapping charge states in the recorded mass spectra (as shown in FIG. 1, which shows a typical mass spectrum for adeno-associated virus type 5 (AAV-5) capsid ions, obtained using more conventional techniques, as described in further detail below).

The Applicants thus recognise that there is room for improved methods for processing ion mobility-mass spectrometry data in order to determine collision cross-sections of ions that have been separated within a drift tube ion mobility spectrometer.

### SUMMARY

A first aspect of the present disclosure provides a method for determining a collision cross section for an ion species, the method comprising:
determining an ion mobility for the ion species using an ion mobility separation device, wherein the ion mobility is determined for the ion species in a first state;
measuring mass to charge ratios for the ion species using a mass spectrometer, wherein prior to measuring the mass to charge ratios, the ion species in the first state is subject to charge reduction to reduce the average charge of the ions to increase the mass to charge ratio spacings between different charge states for the ion species, the mass to charge ratios thus being measured for the ion species in a second, charge-reduced state;
processing the mass to charge ratios measured for the ion species in the second, charge-reduced state to determine an average mass for the ion species;
using the average mass determined for the ion species from the mass to charge ratios measured in the second, charge-reduced state to determine an average charge for the ion species in the first state, prior to the charge reduction; and
using the average charge determined for the ion species in the first state, together with the average mass determined from the ion species in the second, charge-reduced state and the ion mobility determined for the ion species in the first state, to determine a collision cross section for the ion species in the first state.

According to the present disclosure, an ion mobility is measured for ions in a first state. The first state is preferably a “native” state (e.g., and preferably, a (n unmodified) state in which the ions have not (yet) been subject to any charge reduction or other processing of the ions that may change their conformation). For example, the ion species will typically derive from a sample, with the sample being ionised using an appropriate ionisation source in order to generate the ions that are to be analysed. Preferably the ion mobility separation is then performed on the ions in this state. This is because at this point, the ions have preferably undergone minimal conformational changes, and so the ion mobilities determined in this state are expected to more closely reflect the conformation of the original (un-ionised) compound.

In some more conventional analysis arrangements, the ions may then be subject to mass spectrometric analysis in this same first state. That is, the ions may pass directly from the ion mobility separator to a mass spectrometer. The mass spectrometric analysis may thus be used to determine mass to charge values for the different charge state peaks for the different ions corresponding to the ion species of interest, which information can then be suitably processed (e.g. de-convolved) to determine respective average mass and charge values for the ion species. Once the average mass and charge values are determined, these can then be used together with the recorded average ion mobility value in order to calculate a collision cross section for the ion species, using a known relationship between these parameters (e.g., and preferably, using the Mason-Schamp equation (Equation 2) above).

However, for more complicated molecules, where the ion species may have relatively higher masses and numbers of charges, the resulting mass spectra may then be too difficult to resolve, as the different charge state peaks may be too closely spaced together in the mass to charge ratio dimension for the mass spectrometer to resolve the overlapping peaks. In that case, the broad distribution of overlapping charge state peaks appear as a single, broad peak in the mass spectrum. The lack of resolution between the different charge state peaks means that the mass spectrum cannot easily be de-convolved to determine average mass and charge values for the ion species. In turn, this means that the collision cross section for the ion species cannot be calculated.

In the present disclosure, therefore, prior to performing the mass spectrometric analysis, a charge reduction process is performed on the ions to reduce the average number of charges associated with the ions. The mass spectrometric analysis is then performed on the ions in this second, charge-reduced state. This means that the mass to charge ratio peaks for different charge states are then further spaced apart in the mass spectrum, which allows the mass spectrum to be de-convolved to determine the average mass for the ion species.

Thus, in the present disclosure, the mass to charge values are determined for ions in such a second, charge-reduced state. In contrast, the mobility separation is performed for ions in the first state, prior to the charge reduction.

In order to then determine the collision cross section from the ion mobility values that were determined for ions in the first state, it is thus necessary to determine the average charge for the ions in the first state. However, the present disclosure recognises that this can easily be done by determining the average mass for the ion species based on the measurements of the mass to charge values of the ions in the second state, and then using the average mass to determine what the average charge would have been for the ion species in the first state, prior to the charge reduction.

Once the average number of charges for the ion species in the first state has been determined in this way, the average charge state can then be used, together with the ion mobility values (also determined for ions in the first state) and the average ion mass, to determine the collision cross section, e.g. in the normal way, by putting those values into the appropriate equation (e.g., and preferably, the Mason-Schamp equation (Equation 2) above). Thus, in embodiments, determining the collision cross section, CCS, for the ion species uses the (known) equation:

$$K \approx \frac{3}{16} \frac{ze}{N} {( \frac{1}{M} + \frac{1}{m} )}^{1 / 2} {( \frac{1}{{k}_{B} T} )}^{1 / 2} \frac{1}{CCS}$$

where K is the determined ion mobility, is the number of charges on the ion, e is the elementary electric charge, N is the number of gas molecules per unit volume (in the buffer gas), M and m are masses of the ion and the buffer gas molecules, k_{B} is the Boltzmann constant and T corresponds to the temperature of the buffer gas.

The present disclosure thus provides an improved approach for determining collision cross sections for ions that is particularly suitable for high mass high charge complex ions for which the collision cross section could not otherwise easily be determined.

In particular, the above approach allows the ion mobility and collision cross section to be determined for ion species in the first state, without any charge reduction. This is beneficial because the charge reduction process may introduce conformational changes that interfere with the collision cross section values. Thus, the present disclosure recognises that it is desired to be able to measure the collision cross section in the first (e.g. “native”) state, without any charge reduction. However, charge reduction is then separately employed in combination with the mass spectrometry to allow determination of the mass value (which will not be affected by such conformational changes).

The above approach may therefore provide various benefits compared to other possible arrangements.

As mentioned above, according to the present disclosure, the ion mobility separation is performed on the ions in a first state (without charge reduction), whereas the mass spectrometry is performed on the ions in a second state, following a charge reduction. In preferred embodiments, therefore, the ions are passed in sequence from an ion mobility device to a charge reduction device and then onto a mass spectrometer, so that the measurements are all performed in single experimental cycle. This works well because there is preferably no further mobility separation between the end of the ion mobility device and the mass spectrometer, so that even though the ions are recorded by the mass spectrometer in the second, charge-reduced state, the time at which they are recorded is governed by the ion mobility in the first state, prior to the charge reduction.

Thus, in embodiments, the method is implemented in a single combined ion mobility-mass spectrometry apparatus that comprises an ion mobility separation device; a mass spectrometer; and a charge reduction device positioned between the ion mobility separation device and the mass spectrometer, such that ions are passed though the ion mobility separation device in a first state, without charge reduction, whereas ions are passed to the mass spectrometer in a second, charge-reduced state.

In that case, the ion mobility-mass spectrometry apparatus may comprise any other ion processing stages, e.g., that an ion mobility and/or mass spectrometer may desirably contain. For example, the apparatus will typically contain at least an ionisation source for generating ions for analysis. In an embodiment this may comprise an electrospray or nano-electrospray ionisation source, but other arrangements would of course be possible. The apparatus may also contain various other ion-optical components such as ion guides, ion filtering devices, and so on. Correspondingly, the mass spectrometer may comprise any suitable and desired mass analyser. For example, the mass spectrometer may comprise a time of flight mass analyser, but could also comprise, e.g., a quadrupole mass analyser, a triple quadrupole mass analyser, a quadrupole time of flight mass analyser, a quadrupole ion trap, an Orbitrap (RTM), a travelling wave based mass spectrometer, a magnetic and electric sector mass spectrometer, or any combination of these.

However, other arrangements would also be possible. For example, rather than performing the ion mobility and mass spectrometry in sequence, during a single experimental cycle, these measurements could be performed separately, e.g. in successive cycles, or even using two different instruments.

The charge reduction may be performed using any suitable charge reduction process. For example, the charge reduction may use any suitable charge reducing agents. In preferred embodiments, the charge reduction comprises an electron transfer or electron capture process. Thus, in embodiments, the ion mobility-mass spectrometry apparatus (or at least the mass spectrometry apparatus, where the steps may be performed separately) comprises a charge reduction device that comprises an electron transfer (ETD) and/or electron capture (ECD) device. The electron transfer (ETD) and/or electron capture (ECD) device may have any suitable arrangement that such devices may have, as desired. However, other arrangements for performing the charge reduction may be possible, so long as the average charge state can be suitably reduced in the manner of the present disclosure.

In general, the charge reduction process that is used may depend on the polarity of the ions being analysed (which is generally controlled based on the polarity of the ionisation source from which the ions are generated from the sample to be analysed). Thus, when operating in positive ion mode, such that the ion species are positively charged, the charge reduction process should, and preferably does, make the ions less positively charged (to reduce the charge towards zero), e.g. by electron capture (adding electrons or negative charges to the ions). On the other hand, when operating in negative ion mode, such that the ion species are negatively charged, the charge reduction process should correspondingly, and preferably does, make the ions less negatively charged (again, to reduce the charge towards zero), e.g. by electron transfer (removing electrons or adding positive charges to the ions).

Thus, in embodiments, the charge reduction process may be set or selected based on the polarity of the ionisation source. In some embodiments the instrument geometry may be fixed, e.g. so that it always works in positive/negative mode and charge reduction is set accordingly. However, it is also contemplated that the charge reduction process could be switched based on polarity.

The charge reduction preferably reduces the charge state of the ions by a factor of at least two such that the mass to charge ratio spacings between different charge states for the ion species are larger than the widths of mass distributions with a common charge. Preferably, the charge reduction reduces the charge state of the ions by a factor of more than two, such as a factor of at least four.

In preferred embodiments, the ion mobility separation device comprises a (linear) drift tube ion mobility separation device, in which ions are separated through a buffer gas using a uniform (DC) electric field. In that case, the relationship that is used to determine the collision cross section from the average mass, average charge and mobility is a suitable relationship for a drift tube device, e.g., and in particular, the Mason-Schamp equation (Equation 2), presented above. However, in principle, the techniques of the present disclosure may also suitably be applied to other, different types of ion mobility separation device, e.g. by using the determined mobility, mass and charge values in a suitable, different equation relating those parameters for that type of device.

It will be appreciated that within a given sample there may be many ions corresponding to the same ion species. The measured/determined values will therefore typically correspond to ‘average’ values for the ion species that have been determined from the different ions corresponding to that species within the sample that is being analysed. Thus, even if not explicitly stated, it will be appreciated that the ‘mass’, ‘charge’, ‘mobility’, etc., values for the ion species will typically correspond to average values that have been determined using a plurality of ions corresponding to the same ion species.

Correspondingly, within a given sample there may also be many different ions corresponding to different ion species. Accordingly, whilst the method has been described above in relation to determining a collision cross section for a single ion species, it will be appreciated that a given experiment may analyse a sample including a number of different ion species. In that case, the method may correspondingly be performed for multiple (e.g. all) ion species within the sample.

It will be appreciated that the processing of the obtained values will be performed by a suitable data processor (computer). That is, the methods according to the present disclosure are at least in part computer-implemented. The data processor (processing) may be integrated into the analysis apparatus, e.g. as part of an embedded controller that is arranged to process the data. Alternatively, the data generated by the apparatus may be provided to a separate processor or set of processors (including a set of cloud processors) for analysis. Various options are contemplated in this regard.

The present disclosure also extends to methods of processing data that has been previously obtained as such. For instance, the experimental data may be recorded in one place but then processed at a later point, e.g. in an “offline” manner.

Thus, from another aspect of the present disclosure, there is provided a method for determining a collision cross section for an ion species, the method comprising:
obtaining an ion mobility for the ion species, wherein the ion mobility has been determined using an ion mobility separation device for the ion species in a first state;
obtaining mass to charge ratios for the ion species, wherein the mass to charge ratios have been measured using a mass spectrometer, and wherein prior to measuring the mass to charge ratios, ions in the first state have been subject to charge reduction to reduce the average charge of the ions to increase the mass to charge ratio spacings between different charge states for the ion species, the mass to charge ratio thus being measured for the ion species in a second, charge-reduced state;
processing the mass to charge ratios measured for the ion species in the second, charge-reduced state to determine an average mass for the ion species;
using the average mass for the ion species determined from the mass to charge ratios measured in the second, charge-reduced state to determine an average charge for the ion species in the first state, prior to the charge reduction; and
using the average charge determined for the ion species in the first state, together with the average mass determined from the ion species in the second, charge-reduced state and the ion mobility determined for the ion species in the first state, to determine a collision cross section for the ion species in the first state.

In this case, the ion mobility and mass to charge ratios may be obtained in any suitable way. For example, these may be directly obtained from an apparatus. However, these may also be obtained from suitable storage. For instance, the apparatus may generate data that is then written out to appropriate storage. The analysis may then be performed by a processor obtaining the data from the storage and then processing it accordingly.

The methods in accordance with the present technology may thus be implemented at least partially using software e.g. computer programs. It will thus be seen that when viewed from further aspects the present invention provides computer software specifically adapted to carry out the methods herein described when installed on data processing means, a computer program element comprising computer software code portions for performing the methods herein described when the program element is run on data processing means, and a computer program comprising code means adapted to perform all the steps of a method or of the methods herein described when the program is run on a data processing system. The data processing system may be a microprocessor, a programmable FPGA (Field Programmable Gate Array), or any other suitable system.

The technology also extends to a computer software carrier comprising such software which when used to operate a graphics processor, renderer or microprocessor system comprising data processing means causes in conjunction with said data processing means said processor, renderer or system to carry out the steps of the methods of the present invention. Such a computer software carrier could be a physical storage medium such as a ROM chip, CD ROM, RAM, flash memory, or disk, or could be a signal such as an electronic signal over wires, an optical signal or a radio signal such as to a satellite or the like.

It will further be appreciated that not all steps of the methods of the invention need be carried out by computer software and thus from a further broad aspect the present technology provides computer software and such software installed on a computer software carrier for carrying out at least one of the steps of the methods set out herein.

The present technology may accordingly suitably be embodied as a computer program product for use with a computer system. Such an implementation may comprise a series of computer readable instructions either fixed on a tangible medium, such as a non-transitory computer readable medium, for example, diskette, CD ROM, ROM, RAM, flash memory, or hard disk. It could also comprise a series of computer readable instructions transmittable to a computer system, via a modem or other interface device, either over a tangible medium, including but not limited to optical or analogue communications lines, or intangibly using wireless techniques, including but not limited to microwave, infrared or other transmission techniques. The series of computer readable instructions embodies all or part of the functionality previously described herein.

Those skilled in the art will appreciate that such computer readable instructions can be written in a number of programming languages for use with many computer architectures or operating systems. Further, such instructions may be stored using any memory technology, present or future, including but not limited to, semiconductor, magnetic, or optical, or transmitted using any communications technology, present or future, including but not limited to optical, infrared, or microwave. It is contemplated that such a computer program product may be distributed as a removable medium with accompanying printed or electronic documentation, for example, shrink wrapped software, pre-loaded with a computer system, for example, on a system ROM or fixed disk, or distributed from a server or electronic bulletin board over a network, for example, the Internet or World Wide Web.

The present disclosure also extends to an ion mobility-mass spectrometry instrument that is operated in this way.

Thus, from a further aspect of the present disclosure, there is provided an ion mobility-mass spectrometer apparatus comprising:
an ion mobility separation device;
a mass spectrometer; and
a charge reduction device positioned between the ion mobility separation device and the mass spectrometer, such that ions are passed though the ion mobility separation device in a first state, without charge reduction, whereas ions are passed to the mass spectrometer in a second, charge-reduced state;
the apparatus further comprising:
a controller that is configured to:
determine an ion mobility for the ion species using the ion mobility separation device, wherein the ion mobility is determined for the ion species in the first state, without charge reduction;
measure mass to charge ratios for the ion species using the mass spectrometer, the mass to charge ratio being measured for the ion species in the second, charge-reduced state;
process the mass to charge ratios measured for the ion species in the second, charge-reduced state to determine an average mass for the ion species;
use the average mass for the ion species determined from the mass to charge ratios measured in the second, charge-reduced state to determine an average charge for the ion species in the first state, prior to the charge reduction; and
use the average charge determined for the ion species in the first state, together with the average mass determined from the ion species in the second, charge-reduced state and the ion mobility determined for the ion species in the first state, to determine a collision cross section for the ion species in the first state.

It will be appreciated that the present invention in any of these further aspects may include any or all of the features described in relation to the first aspect of the invention, and vice versa, at least to the extent that they are not mutually inconsistent. It will also be appreciated by those skilled in the art that all of the described embodiments of the invention described herein may include, as appropriate, any one or more or all of the features described herein.

As mentioned above, the techniques described above may find particular utility for ion species that would otherwise (without charge reduction) be expected to contain large numbers of overlapping charge state peaks, that could not easily be resolved. Examples of this may be protein complexes such as virus capsids, in particular an adeno-associated virus (AAV) capsids. These are clinically important molecules and the present disclosure facilitates analysis of these, and other similarly complex, molecules. However, the present disclosure may of course be used to analyse any suitable compounds or species, as desired.

The present Applicants have recognised that the use of charge reduction to increase the mass to charge ratio spacings between different charge states may be beneficial in its own right to enable the mass determination of high molecular weight species independently of whether ion mobility separation is employed.

Thus, a further aspect of the present disclosure provides a method for determining a mass value for an ion species, the method comprising:
measuring mass to charge ratios for the ion species using a mass spectrometer, wherein prior to measuring the mass to charge ratios, the ion species in the first state is subject to charge reduction to reduce the average charge of the ions to increase the mass to charge ratio spacings between different charge states for the ion species, the mass to charge ratios thus being measured for the ion species in a charge-reduced state;
processing the mass to charge ratios measured for the ion species in the charge-reduced state to determine a mass value for the ion species.

A further aspect still of the present disclosure provides a mass spectrometer apparatus comprising:
a mass spectrometer; and
a charge reduction device positioned upstream of the mass spectrometer, such that ions are passed to the mass spectrometer in a charge-reduced state;
the apparatus further comprising:
a controller that is configured to:
process the mass to charge ratios measured for the ion species in the charge-reduced state to determine a mass value for the ion species.

The determination of the mass values according to these further aspects may be performed in any suitable and desired manner. In an embodiment, this is done using an expected or modelled mass distribution for the ion species. For example, and preferably, processing the mass to charge ratios measured for the ion species in the charge-reduced state to determine a mass value for the ion species comprises performing a maximum entropy deconvolution.

It will be appreciated that the present invention in any of these further aspects may include any or all of the features described in relation to the first aspect of the invention, and vice versa, at least to the extent that they are not mutually inconsistent. It will also be appreciated by those skilled in the art that all of the described embodiments of the invention described herein may include, as appropriate, any one or more or all of the features described herein.

Thus, in embodiments, the charge reduction process comprises electron transfer and/or electron capture. Preferably, the charge reduction reduces the charge state of ions by a factor of at least two, and preferably by a factor of more than two, such as a factor of at least four.

Preferably, the ion species that are analysed in these further aspects correspond to high molecular weight (>1 MDa) species, for example, such as protein complexes such as virus capsids, in particular an adeno-associated virus (AAV) capsids, as described above. The mass spectrometer according to these further aspects may comprise any suitable and desired mass spectrometer. For example, in an embodiment, the mass analyser comprises a time of flight analyser. However, other arrangements would be possible, as discussed above, and in general the mass spectrometer may comprise any suitable and desired components that a mass spectrometer may have.

### BRIEF DESCRIPTION OF THE DRAWINGS

Various embodiments will now be described, by way of example only, and with reference to the accompanying drawings in which:

FIG. 1 shows an example of a typical mass spectrum for adeno-associated virus type 5 (AAV-5) capsid ions obtained using more conventional mass spectrometry;

FIG. 2 shows an example of a mass spectrum for adeno-associated virus type 5 (AAV-5) capsid ions obtained according to an embodiment of the present disclosure wherein the ions have been subject to charge reduction, resulting in a reduction in the average charge state and hence an increased spacing in mass to charge ratio;

FIG. 3 shows a schematic of an example of an analytical instrument that can be used in accordance with embodiments of the present disclosure;

FIG. 4 is a flowchart showing a method according to an embodiment of the present disclosure; and

FIG. 5 shows spectra of empty AAV5 capsids recorded following interaction with electrons inside an electron capture device (ECD).

### DETAILED DESCRIPTION

The present disclosure relates to improved techniques for determining ion collision cross section using drift tube ion mobility spectrometry. Collision cross section between neutral and ion pair is an analytical metric indicative of conformational preferences of the molecule. It is typically determined using apparatus consisting of an ion mobility cell and mass spectrometer.

In the ion mobility cell, a packet of ions drifts (moves at terminal velocity) through a buffer gas under the influence of an electric field. This relationship is described by Equation 1, above. Subsequent to the ion mobility cell, the ion's mass-to-charge ratio (m/z) is then measured in a mass spectrometer. The mass-to-charge ratio (m/z) can then be processed (de-convolved) in order to extract the average mass and charge values for the ion species of interest. The average mass and charge values, together with the average ion mobility (K) as determined by the time of detection, can then be input into the Mason-Schamp equation (Equation 2, above) in order to calculate the average collision cross section for the ion species.

However, there are situations where heterogeneity in ion mass, relatively high number of charges or the insufficient resolution of a mass spectrometer preclude the deconvolution of mass and charge from MS data. This impedes the use of the formula above to determine an ion's CCS. One such situation exists for multiply charged ions of high mass protein complexes such as virus capsids.

Such virus capsids may be of significant clinical interest. For example, adeno-associated virus (AAV) capsids are an attractive candidate for gene therapy vectors. These ball-like particles consist of 60 subunits of three viral proteins (VP1, VP2 and VP3) assembled in a variety of stoichiometries (expected average VP ratio of 1:1:10), giving an expected average mass ˜3.7 MDa. “Full” capsids encapsulate ˜4.7 kb single-stranded DNA. These are therefore relatively large molecules that when ionised for mass spectrometry typically contain large numbers of overlapping charge states.

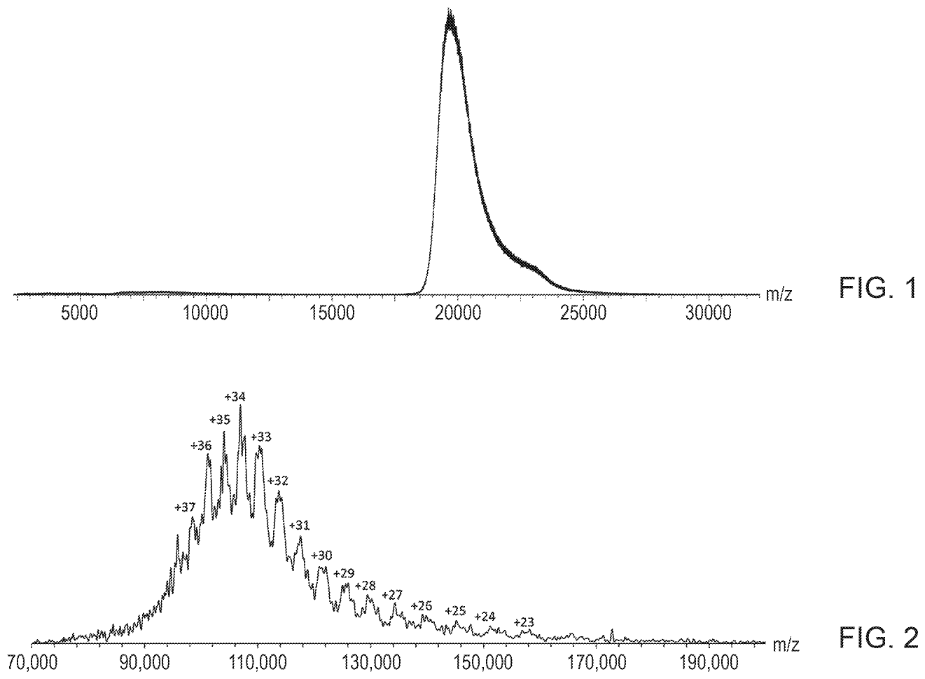

FIG. 1 is a mass spectrum of AAV-5 capsid ions generated in an electrospray ionisation source in positive polarity using conventional mass spectrometry. The mass spectrum shown in FIG. 1 features a single broad profile, composed of many overlapping charge state peaks. Thus, although the mass to charge ratio can be derived, the lack of fine isotopic and charge state structure precludes deconvolution of mass and charge information. In turn, this means that the collision cross section cannot be directly determined in the manner described above.

Thus, precise determination of capsid mass remains challenging due to the high level of adduction and heterogeneity in the VP protein ratio which yields broad distributions of overlapping charge states (as shown in FIG. 1).

The present disclosure recognises that to address this problem, the ions may be subjected to a charge reduction process, resulting in the reduction of the average charge state, and hence increasing the spacing between different charge states. FIG. 2 is a corresponding mass spectrum of AAV-5 ions generated using an electrospray ionisation in positive polarity but wherein prior to the mass analysis the ions were subjected to interactions with electrons in an electron capture device to reduce the average charge state. In contrast to FIG. 1, FIG. 2 features distinct peaks with increasing mass to charge value spacing, characteristic of a charge state distribution. Thus, it is possible to de-convolve mass and charge information. From the data above it can be inferred that the average mass is ˜3.6 MDa. The resolved charge state peaks in FIG. 2 are annotated based on the de-convolved mass of 3.6 MDa.

Performing charge reduction in this way can therefore allow the mass values to be determined, even for complex biomolecules such as AAV-5. However, it is often the case that an ion's conformation is influenced by coulombic repulsions between charged residues. On the other hand, the structure of ions generated from native solution conditions more closely resembles those in-vivo. In order to properly characterise and identify ions it is therefore of interest to measure the collision cross sections of ions in their “native” charge state, without any charge reduction.

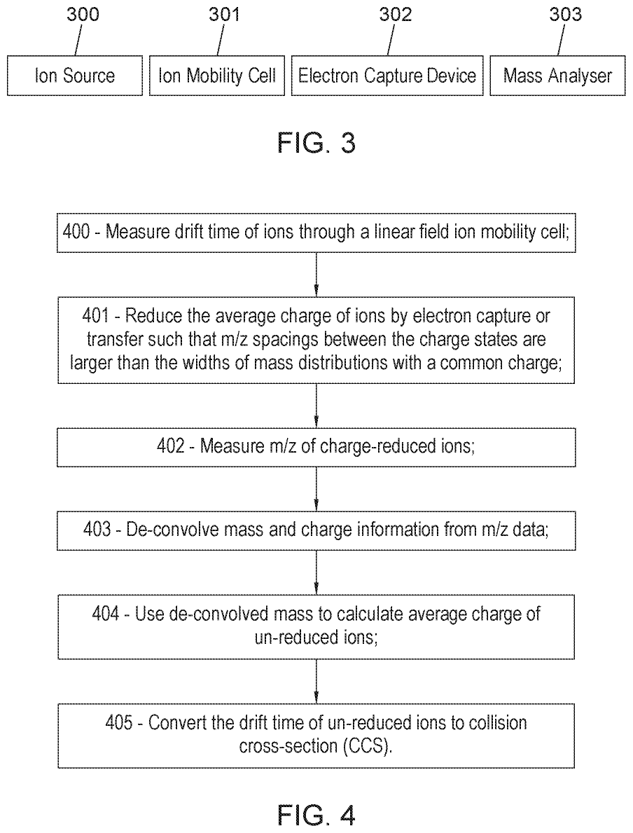

FIG. 3 shows a schematic of an example of an analytical instrument that can be used in accordance with embodiments of the present disclosure. As shown in FIG. 3, the instrument comprises an ionisation source 300 through which a sample can be introduced. The ionisation source 300 may comprise any suitable ionisation source. For example, in an embodiment, it may be comprise an electrospray ionisation source. Electrospray ionisation sources are well-suited to analysis of complex biomolecules (but typically generate lots of different charge states). In this example the ionisation source 300 is operated in positive polarity mode.

The ions generated by the ionisation source 300 are then passed to an ion mobility cell 301. In the present embodiment the ion mobility cell 301 comprises a linear drift tube ion mobility device.

An electron capture device (ECD) 302 is then installed downstream of the ion mobility cell 301. The ions exiting the ion mobility cell 301 thus pass through a cloud of electrons within the electron capture device (ECD) 302 in order to reduce the average charge (since in this example the ionisation source 300 is operated in positive polarity mode). The charge-reduced ions are then passed to a mass analyser 303 which in an example comprises a time-of-flight mass analyser but any other suitable mass analyser may be used, as desired.

The resulting mass spectra generated by a mass analyser 303 will thus correspond to mass spectra like that shown in FIG. 2 where there is an increased spacing between different charge state peaks (due to the charge reduction). Since no mobility separation takes place between the end of the ion mobility cell and mass analyser, all charge-reduced ions will present with the same average drift time, corresponding to that of their un-reduced (native) precursors.

In order to determine the collision cross section from the recorded mass spectrum, it is therefore necessary to determine the average number of charges for the ion species in the native state (e.g. corresponding to the data shown in FIG. 1), i.e. in the state it was in during the ion mobility separation. The information derived from FIG. 2 above allows determining that the average number of charges on un-reduced ions is 3.6e6/22,000≈164. That is, the average charge in the native state can be calculated based on the average mass to charge value in the native state and the average mass as determined from FIG. 2. These values can then be used together with the ion's mobility determined based on the average drift time determined using the ion mobility cell to calculate the collision cross-section using the Mason-Schamp equation (Equation 2).

Thus, in embodiments, the present disclosure provides an improved method of determining collision cross section of multiply charged ions which would when analysed using more conventional techniques include unresolved charge state peaks that prevent the precise mass value being determined and thus prevent the collision cross section from being determined in the normal way. In particular, collision cross sections can be determined for ions in their “native” charge state (without any charge reduction). However, charge reduction is then applied to allow precise mass determination, which mass value can then be used to calculate the average charge in the native state, and hence also the collisional cross section, in the normal way.

FIG. 4 is a flowchart showing the overall method according to the present embodiment. The method comprises the following steps: (400) measuring drift time of ions through a linear field ion mobility cell; (401) reducing the average charge of ions by electron capture or transfer such that m/z spacings between the charge states are larger than the widths of mass distributions with a common charge; (402) measuring m/z of charge-reduced ions; (403) de-convolving mass and charge information from m/z data; (404) using deconvolved mass to calculate the average charge of un-reduced ions; and (405) converting the drift time of un-reduced ions to collision cross section using the Mason-Schamp equation (Equation 2).

Whilst an embodiment is described above in which mass spectrometry follows ion mobility separation within a single analytical instrument (as shown in FIG. 3), such that the ion mobility and mass spectrometry measurements are performed during a single experimental cycle, it will be appreciated that the ion mobility and mass spectrometry measurements may also be performed in separate experimental cycles.

Correspondingly, whilst in the example above, electron capture is used to perform charge reduction, it would also be possible to use other charge reduction processes, e.g., and in particular, depending on the polarity of the ionisation source 300. For example, in negative mode, where the ions are negatively charged, it may be possible to reduce the charge (towards zero) using an electron transfer process, where electrons are removed from the ions (or positive charges are added) to make the ions less negative. Other suitable charge reduction processes may also be used, as desired.

It is also recognised that the use of charge reduction may enable mass determination of high molecular weight species such as AAVs using Time of Flight mass spectrometry independently of whether ion mobility separation is employed. Thus, in embodiments, the ion mobility cell 301 may be omitted from FIG. 3. In that case, rather than applying the techniques described above in order to determine a collision cross section of ions, the measured m/z values of the charge-reduced ions by the mass analyser 303 may be processed to determine mass values.

For instance, it has been found that the AAV assembly process can result in a mixture of particles with up to 1891 different viral protein (VP) ratios (i.e. masses). Each species is expected to present in a range of charge states, resulting in a crowded m/z distribution. Even a moderate amount of adduction can “blur” such m/z profiles making simultaneous deconvolution of all underlying masses extremely challenging.

Using charge reduction as described above however provides one way to simplify crowded m/z profiles of AAVs as the charge reduction can reduce the average charge such that m/z spacings between the charge states are larger than the widths of mass distributions with a common charge.

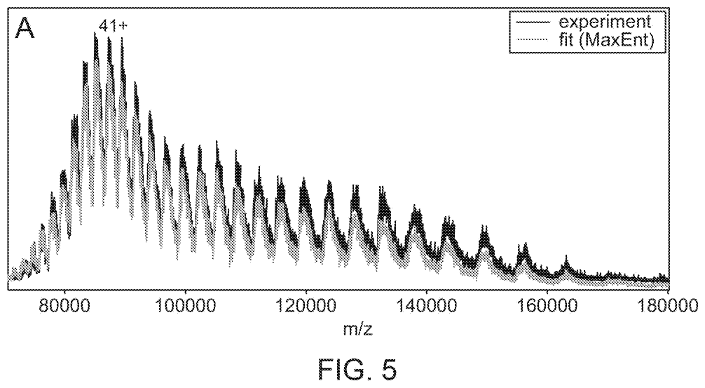

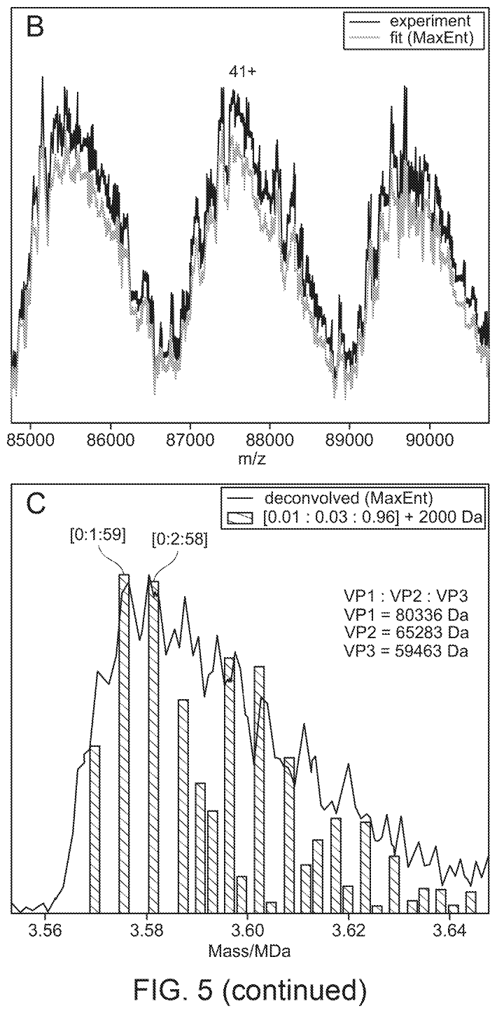

FIG. 5 shows spectra of empty AAV5 capsids recorded following interaction with electrons inside an electron capture device (ECD) 302. By passing the AAV5 ions through the electron capture device (ECD) 302 a significant m/z shift is observed (from m/z˜20,000 to ˜90,000) compared to the spectrum of empty AAV5 capsids without charge reduction, and distinct peaks with increasing m/z spacing, characteristic of a charge state distribution, appear in the recorded spectrum, as shown in FIG. 5. In particular, the upper (darker) trace in FIG. 5, panel A shows the experimental spectra.

Using a modified version of the MaxEnt 1 algorithm, the experimental m/z data were deconvolved to the corresponding mass and charge components, as shown in FIG. 5, panel C. As shown here, the most abundant mass was determined to be at ˜3.580 MDa. This is somewhat smaller than the expected value of ˜3.7 MDa perhaps due to the altered ratio of VP proteins, as discussed below. Notably, the deconvolved mass profile is asymmetric, which again may be explained by some of the lighter assemblies being the most abundant ones.

The fit resulting from the deconvolved data is also shown in FIG. 5, panel A, as the lower (lighter) trace. FIG. 5, panel B shows a zoomed in portion of the spectrum in the region from m/z˜85,000 to ˜90,000 again showing the experimental data (upper, darker trace) and the fit resulting from the deconvolved data (lower, lighter trace).

In order to deconvolve the data, the assembly process of capsids was proposed to be stochastic, with the probability of formation of “different-ratio” capsids approximated by a multinomial distribution, where multinomial probabilities reflect the VP expression levels, in solution. The deconvolved data was then fitted with multinomial distribution, constrained to the total of 60 samples (i.e. 60 VPs in a capsid).

Using this approach, the “best fit” multinomial probabilities were found at [0.01:0.03:0.96], accounting for 2000 Da mass shift due to adduction (FIG. 5, panel C, grey bars vs the trace). The two most probable masses predicted by the above method (3.576 and 3.581 MDa) correspond to capsids with VP ratios of [0:1:59] and [0:2:58], respectively. The spacing between the most abundant peaks is ˜5000 Da which is significantly higher than the estimated amount of adduction so it may be possible that some of the features in the deconvolved mass distribution are “real”. Nonetheless, FIG. 5 shows that this approach can work to determine mass values even for high molecular weight species.

The resulting mass profile may further be convolved with charge state distribution to model the resonance pattern in the “native” m/z distribution. Finally, it is noted that the observed asymmetric mass distribution may be caused by the biological/process artefact (e.g. “correlated” assembly, mixed batches), in which case a mix of multinomial (or other) distributions may be more appropriate to describe the data. The technique described above can easily be extended in this way.

Thus, in an embodiment, there is provided a method for determining a mass value for an AAV virus capsid that is performed using an apparatus like that shown in FIG. 3 but without necessarily including an ion mobility cell 301 (although one may be provided if desired) the method comprising performing electron capture charge reduction to reduce the charge state of the capsid ions by at least a factor of two, and then measuring the m/z values using the mass analyser 303. A maximum entropy deconvolution process can then be performed on the resulting m/z values, e.g. using MaxEnt algorithm, to determine the mass of the AAV capsid.

In this case, the method preferably comprises:
1. Spraying and ionising virus capsids (e.g. from ammonium acetate solution (50-1000 mM)) by electrospray or nano-electrospray process.
2. Allowing interaction of capsid ions with electrons (in e.g. ECD, ETD) or with charge reducing agents.
3. Reducing the charge state of capsid ions by a factor of 2, such that the m/z spacings between the neighbouring charge state peaks are larger than the widths of mass distributions with a common charge.
4. Measuring the m/z of charge reduced ions using a mass spectrometer such as a time of flight mass spectrometer (ToF-MS), a quadrupole mass spectrometer (Quad-MS), a triple quadrupole mass spectrometer (QQQ), a quadrupole time of flight mass spectrometer (Q-ToF), an Orbitrap (RTM), a quadrupole ion trap (QIT) (all other MS), a travelling wave based mass spectrometer (MS), and a magnetic and electric sector mass spectrometer.
5. Inferring mass and charge information from the m/z data produced (for example, MaxEnt or BayesSpray deconvolution).

The embodiments described above thus facilitate obtaining more meaningful data from the analysis of species that would otherwise be too complex to analyse. The techniques above are thus particularly suitable for analysing virus capsids with molecular weights in excess of 1 MDa. This may include, for example:
Capsids of adeno-associated virus (AAV).
Capsids consisting of various amounts of various viral protein constituents.
Capsids consisting of at least 60 viral proteins where there are three possible viral proteins of distinct molecular weights (VP1, VP2, VP3).
Where assembly process result in varying ratio of VP1:VP2:VP3.
Where mass distribution of assembled capsids is heterogenous due to heterogeneity in VP ratio.
Where electrospray ionisation of capsids results in crowded m/z distribution of overlapping charge state peaks.

Various other arrangements would of course also be possible. Thus, although the present disclosure has been described with reference to various embodiments, it will be understood by those skilled in the art that various changes in form and detail may be made without departing from the scope defined by the accompanying claims.

1. The method of claim 6, wherein the step of obtaining an average ion mobility for the ion species comprises determining an average ion mobility using an ion mobility separation device and wherein the step of obtaining mass to charge ratios for the ion species consists of measuring the mass to charge ratios using a mass spectrometer.

2. The method of claim 1, wherein the ion mobility separation device comprises a linear drift tube ion mobility separation device.

3. The method of claim 1, wherein the charge reduction process comprises electron transfer and/or electron capture.

4. The method of any of claim 1, wherein the ion species is generated from a sample by an ionisation source, and wherein the charge reduction process is set or selected based on the polarity of the ionisation source.

5. The method of claim 2, wherein the ions are passed in sequence from the ion mobility device to a charge reduction device and then onto the mass spectrometer, so that the measurements for determining ion mobility and mass to charge ratios are performed in single experimental cycle.

6. A method for determining a collision cross section for an ion species, the method comprising:
obtaining an ion mobility for the ion species, wherein the ion mobility has been determined using an ion mobility separation device for the ion species in a first state;
obtaining mass to charge ratios for the ion species, wherein the mass to charge ratios have been measured using a mass spectrometer, and wherein prior to measuring the mass to charge ratios, ions in the first state have been subject to charge reduction to reduce the average charge of the ions to increase the mass to charge ratio spacings between different charge states for the ion species, the mass to charge ratio thus being measured for the ion species in a second, charge-reduced state;
processing the mass to charge ratios measured for the ion species in the second, charge-reduced state to determine an average mass for the ion species;
using the average mass for the ion species determined from the mass to charge ratios measured in the second, charge-reduced state to determine an average charge for the ion species in the first state, prior to the charge reduction; and
using the average charge determined for the ion species in the first state, together with the average mass determined from the ion species in the second, charge-reduced state and the ion mobility determined for the ion species in the first state, to determine a collision cross section for the ion species in the first state.

7. A computer program product storing instructions that when executed by a processor will cause the processor to perform a method for determining a collision cross section for an ion species, the method comprising:
obtaining an ion mobility for the ion species, wherein the ion mobility has been determined using an ion mobility separation device for the ion species in a first state;
obtaining mass to charge ratios for the ion species, wherein the mass to charge ratios have been measured using a mass spectrometer, and wherein prior to measuring the mass to charge ratios, ions in the first state have been subject to charge reduction to reduce the average charge of the ions to increase the mass to charge ratio spacings between different charge states for the ion species, the mass to charge ratio thus being measured for the ion species in a second, charge-reduced state;
processing the mass to charge ratios measured for the ion species in the second, charge-reduced state to determine an average mass for the ion species;
using the average mass for the ion species determined from the mass to charge ratios measured in the second, charge-reduced state to determine an average charge for the ion species in the first state, prior to the charge reduction; and
using the average charge determined for the ion species in the first state, together with the average mass determined from the ion species in the second, charge-reduced state and the ion mobility determined for the ion species in the first state, to determine a collision cross section for the ion species in the first state.

8. An ion mobility-mass spectrometer apparatus comprising:
an ion mobility separation device;
a mass spectrometer; and
a charge reduction device positioned between the ion mobility separation device and the mass spectrometer, such that ions are passed though the ion mobility separation device in a first state, without charge reduction, whereas ions are passed to the mass spectrometer in a second, charge-reduced state;
the apparatus further comprising:
a controller that is configured to:
determine an ion mobility for the ion species using the ion mobility separation device, wherein the ion mobility is determined for the ion species in the first state, without charge reduction;
measure mass to charge ratios for the ion species using the mass spectrometer, the mass to charge ratio being measured for the ion species in the second, charge-reduced state;
process the mass to charge ratios measured for the ion species in the second, charge-reduced state to determine an average mass for the ion species;
use the average mass for the ion species determined from the mass to charge ratios measured in the second, charge-reduced state to determine an average charge for the ion species in the first state, prior to the charge reduction; and
use the average charge determined for the ion species in the first state, together with the average mass determined from the ion species in the second, charge-reduced state and the ion mobility determined for the ion species in the first state, to determine a collision cross section for the ion species in the first state.

9. The apparatus of claim 8, wherein the ion mobility separation device comprises a linear drift tube ion mobility separation device.

10. The apparatus of claim 8, wherein the charge reduction device comprises an electron transfer and/or electron capture device.

11. The apparatus of any of claim 8, further comprising an ionisation source for generating the ion species from a sample to be analysed, wherein the charge reduction process is set or selected based on the polarity of the ionisation source.

12-21. (canceled)
